(19) United States
(12) Patent Application Publication
(10) Pub. No.: US 2013/0211320 A1
(43) Pub. Date: Aug. 15, 2013

(54) SYSTEM AND METHOD FOR DELIVERING AN ANTI-ADHESIVE SUBSTANCE TO A BODY CAVITY

(71) Applicants: Nawar Alkhamesi, London (GB); Robert R. Mantell, Arlington Heights, IL (US)

(72) Inventors: Nawar Alkhamesi, London (GB); Robert R. Mantell, Arlington Heights, IL (US)

(21) Appl. No.: 13/840,460

(22) Filed: Mar. 15, 2013

Related U.S. Application Data
(63) Continuation-in-part of application No. 13/590,953, filed on Aug. 21, 2012.
Application No. 13/590,953 is a continuation of application No. 13/336,163, filed on Dec. 23, 2011.
Application No. 13/336,163 is a continuation of application No. 12/726,137, filed on Mar. 17, 2010, now Pat. No. 8,105,267.
Application No. 12/726,137 is a continuation of application No. 10/961,475, filed on Oct. 7, 2004, now Pat. No. 7,704,223.
(60) Provisional application No. 60/509,733, filed on Oct. 7, 2003.

(51) Int. Cl. A61M 13/00

(52) U.S. Cl. CPC A61M 13/003
USPC 604/24

(57) ABSTRACT

A system and method for creating a medicated atmosphere in an organ, or body cavity is disclosed. The system includes a flexible nebulization catheter that can be manipulated during use, a device for the introduction of the nebulization catheter, a medication delivery apparatus configured to control delivery of a medication to the catheter, a gas delivery apparatus in communication with the catheter, a gas pressure relief apparatus configured to relieve pressure in the organ or body cavity, and a central controller. The system may include a liquid source having a mixture of hyaluronic acid and heparin that, in conjunction with the nebulizing catheter, are designed to generate an aerosol with a particle size of 10-25 microns in a body cavity. The method includes providing an aerosol of anti-adhesive medication to an organ or body cavity while controlling overall pressure in the organ or cavity.

### CROSS-REFERENCE TO RELATED APPLICATION

This application is a continuation-in-part of Application No. 13/590,953, filed Aug. 21, 2012, pending, which is a continuation of application Ser. No. 13/336,163, filed Dec. 23, 2011, pending, which is a continuation of application Ser. No. 12/726,137, filed Mar. 17, 2010, now U.S. Pat. No. 8,105,267, which is a continuation of application Ser. No. 10/961,475, filed Oct. 7, 2004, now U.S. Pat. No. 7,704,223, which claims the benefit of U.S. Provisional Application No. 60/509,733, filed Oct. 7, 2003, and the entirety of each of these applications is incorporated herein by reference.

### FIELD OF THE INVENTION

The present invention relates to a system and method for delivering a substance to a body cavity. More particularly, the present invention relates to a system and method for delivering an anti-adhesive substance or mixture to a body cavity in conjunction with a minimally invasive operative procedure or for therapeutic treatment unrelated to a surgical procedure.

### BACKGROUND

Among problems that physicians have encountered during diagnostic or surgical procedures, using both “open” techniques, and minimally invasive (laparoscopic) surgical techniques, are numerous post procedural complications. These complications can consist of, but are not limited to, post operative pain, infections, tissue adhesions, and tumor formation. Numerous products, such as medications and associated delivery systems, addressing these issues exist on the market to improve the surgical or invasive experience and patient outcomes. Among these products are suction and irrigation wands that are used for flushing tissue sites with sterile water or saline and removing blood. There are medications, which are spread over exposed organs, to coat or provide a barrier between tissue and organs for prevention of adhesions. These materials may be in gel form, sheet form, spray (liquid) form, or aerosol form to coat organs or tissues, or to provide thin layer deposition to the organs in the operative site. Some of these materials may be used in both open and minimally invasive surgical techniques.

The problems with these materials, and their application as related to laparoscopy, are their inability to be used easily and effectively in a minimally invasive laparoscopic environment. Among the difficulties associated with spraying of liquids, is the pooling and lack of containment of the fluids used with irrigation and aspiration wands. It is also difficult to cover large areas (greater than several square centimeters), and do so without using much more medicament than is necessary. This contributes to the cost of excessive medication, and adding to the cost and time of the surgery.

Materials used in sheet form are not practical to apply to the organs when using laparoscopic minimally invasive techniques, due to the difficulty in getting the material through standard trocars, and then spreading the material out over the affected area, and keeping it in place once positioned. The liquid spray technique has many of the same problems as the irrigation approach. These devices normally force a liquid through a cannula like device under pressure. The introduction of additional fluid into the body cavity can cause increases in pressure and do not include a means for pressure relief. Without a means for directing the spray, it is difficult to control where the medication is deposited, and in what amount. Also, the precise disposition of the medication as to amount and location is difficult to control.

Compound materials are sometimes mixed prior to being aerosolized by a hand held syringe device, and then by applying an air stream to the mixed medication as it is being dispensed, to create an aerosolized stream that is used to “paint” the organs. This method also ignores the problem of the creation of additional pressure in the organ with no relief mechanism. Creating an aerosol “cloud” contends with the problem of how to effectively coat all the surfaces required, but also introduces the problem of increasing abdominal pressures uncontrollably inside an insufflated body cavity or organ, such as the peritoneum.

All of the above methodologies, while focused on applying substances in different physical forms for the purpose of treating or coating tissues and/or organs, have not been optimized for use in the laparoscopic, minimally invasive environment. The term “substance”, as used in this specification, includes, without limitation, a liquid, powder or gas, or any combination thereof.

### BRIEF SUMMARY

In order to address the deficiencies in the prior art, a system and method for providing a substance to a body cavity to minimize adhesions is discussed below. According to a first aspect of the invention, a system is provided that will allow the application of a substance, such as an aerosolized medicament to a distended body cavity that will allow for the efficient, safe, and effective application of any number of substances, such as a mixture of hyaluronic acid and heparin, to prevent tissue adhesion. The system may include a source of pressurized gas, a source of liquid, and a nebulizing catheter. The nebulizing catheter may include at least one liquid lumen and at least one gas lumen, where the at least one liquid lumen is in communication with the source of liquid and the at least one gas lumen is in communication with the source of pressurized gas. The nebulizing catheter has a distal end positionable in a body cavity and the liquid lumen and gas lumens are oriented at the distal end to mix the gas and liquid to generate an aerosol inside the body cavity to cover exposed organs and a wall of an abdomen. The liquid comprises a mixture of hyaluronic acid and heparin in a ratio by volume in the range of 1:1 to 1:10 of hyaluronic acid to heparin.

According to another aspect of this invention, the catheter may include one liquid lumen and a plurality of gas lumens sized and oriented at the distal end to mix the gas and liquid to generate an aerosol having a particle size in a range of 10 microns to 25 microns inside the body cavity to cover exposed organs and a wall of an abdomen. A liquid and gas dispensing controller is configured to manage delivery to the nebulizing catheter of gas from the source of pressurized gas and liquid from the source of liquid, and the liquid is a mixture of hyaluronic acid and heparin. The hyaluronic acid may have a molecular weight in a range of 600 kilo Daltons (kDa) to 4,000 kDa, in one implementation, or in a range of 1,000 kDa up to 2,000 kDa in another.

Further aspects and advantages of the invention are discussed below in conjunction with the preferred embodiments.

### BRIEF DESCRIPTION OF THE DRAWINGS

FIG. 1 illustrates an embodiment of fluid connections in a system for laparoscopic delivery of aerosolized medication according to one embodiment of the present invention.

FIG. 2 is a perspective view of a nebulizing catheter suitable for use in the system of FIG. 1.

FIG. 3 is an alternative embodiment of the nebulizing catheter of FIG. 2.

FIG. 4 is an alternative embodiment of the system of FIG. 1.

FIG. 5 is a schematic view of an embodiment of control connections of the system of FIG. 1.

FIG. 6 is a block diagram of a regulated liquid and gas dispensing controller suitable for use in the system of FIG. 1.

FIG. 7 is a block diagram of an alternative embodiment of the regulated liquid and gas dispensing controller of FIG. 6 having a fluid mixing chamber for dispensing and mixing multiple fluids.

FIG. 8 is a block diagram of a second alternative embodiment of the regulated liquid and gas dispensing controller of FIG. 6 having a y-tube for dispensing and mixing multiple fluids.

FIG. 9 is a block diagram of a third alternative embodiment of the regulated liquid and gas dispensing controller of FIG. 6 having a gas mixing chamber for providing a mixed insufflation gas.

FIG. 10 is a block diagram of a disposable catheter, syringe and tubing set attached to the regulated liquid and gas dispensing controller of FIG. 6.

FIG. 11 is a perspective view of a syringe pump having a receiving slot for a disposable syringe/catheter/tubeset.

FIG. 12 is an exploded view of a system for manipulating a catheter inserted in a body cavity according to an embodiment of the present invention.

FIG. 13 is an end view of an embodiment of the tip of the catheter of the system of FIG. 12.

### DETAILED DESCRIPTION OF THE PRESENTLY PREFERRED EMBODIMENTS

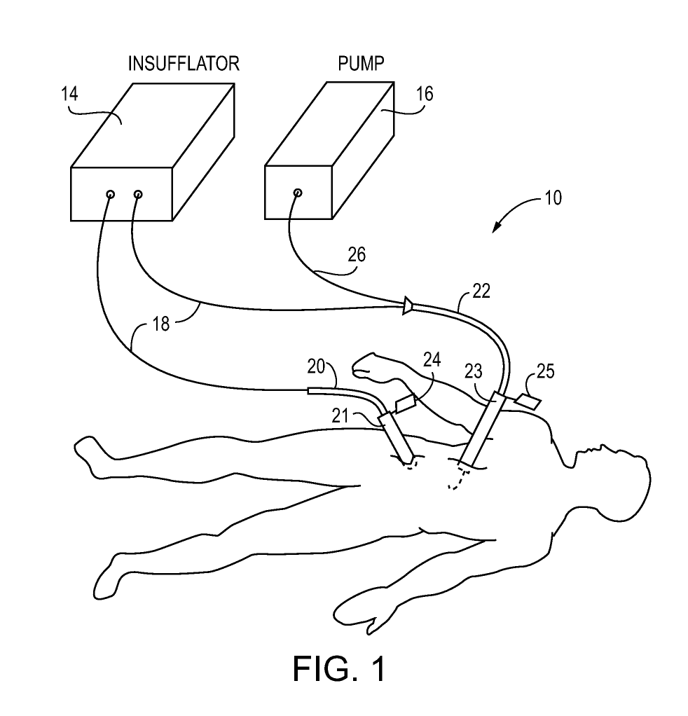

Referring to FIG. 1, an embodiment of a system 10 for delivery of a substance to a body cavity is shown connected to a patient 12. The system 10 includes an insufflator 14 for providing a supply of insufflation gas to the patient 12. The system also includes a pump 16 configured to controllably supply a medicament to the patient 12. The insufflator 14 connects to gas delivery lines 18 and then to one or more catheters 20, 22. The insufflator 14 may include an integrated gas temperature control mechanism or may be combined with one or more in-line gas heaters to control the temperature of gas supplied for insufflation and/or nebulization. A first catheter is an insufflation catheter 20 sized for cooperating with a trocar 21 or other standard catheter insertion needle so that the insufflation catheter may be directed into the peritoneum or other specific location in the patient 12. A pressure relief control valve 24 is positioned along the supply of insufflation gas, for example on the catheter 20 or trocar 21, so that pressure in the peritoneum or other target location in the patient will be monitored and adjusted to maintain a desired level.

An aerosolization gas supply, preferably separately controllable from the general insufflation gas sent through the gas delivery line 18 to the nebulization catheter 22, is also supplied by the insufflator 14. This aerosolization gas supply is directed through a gas line 18 connected to a nebulization catheter 22 inserted into the peritoneum through another trocar 23 or other suitable needle. Although the system 10 may operate with a single pressure relief control valve positioned anywhere along the components making up the insufflation gas supply chain, a separate and independently controllable pressure relief valve 25 may be positioned on the nebulization gas supply, such as at the trocar 23 for the nebulization catheter 22. The nebulization catheter receives a medicament in fluid form from fluid supply line 26 connected with the pump 16. The gas provided to the nebulizing catheter is mixed with a fluid medicament supplied by the pump 16 and generates a nebulized medicament for deposit on specific organs, on the peritoneum cavity wall and other locations within the patient 12. The system of FIG. 1 is shown with only the basic fluid and gas lines for clarity. A central controller, described in greater detail below, coordinates the actions of the insufflator, pump, and pressure relief control valve(s) so that any of the system parameters, such as pressure, gas or fluid flow rate, temperature and so on, may be managed. Although a nebulizing catheter is shown, any of a number of other devices for introducing a substance into a body cavity may also be used. For example, the nebulizing catheter 22 may be replaced by a suction irrigation wand to infuse the body cavity with a substance.

The insufflator 14 may be any of a number of insufflators, such as the OMNIFLATOR Model 6620 available from Northgate Technologies, Inc. of Elgin, Ill. Examples of suitable insufflators are described in U.S. Pat. No. 6,299,592 and U.S. application Ser. No. 10/829,485, and the entirety of each of these references are incorporated by reference. The insufflator may include a pressurized source of insufflation gas. Examples of insufflation gases include, but are not limited to, carbon dioxide, nitrous oxide, argon, or helium. The insufflation gas is typically reduced in pressure by the insufflator to approximately 45 to 55 millimeters of mercury (also know as a “push” pressure), although the pressure may be changed depending on the insufflator in use and any regulations that may be in force. While the push pressure may be in the range of 45-55 millimeters of mercury, the actual pressure maintained in the peritoneum or other body cavity is preferably less than 25-30 mm of mercury and, in the case of many laparoscopic surgeries, most preferably in the range of 12 mm of mercury.

The pump 16 may be a peristaltic pump, syringe pump, hydraulic (air over liquid) pump or any other mechanism capable of controlling the dispensing of medication. Controllable pump parameters may include the rate and volume, as well as the timing, of delivery. It is contemplated that continuous and periodic pumping may be desired. Delayed pumping of medication, such as the transport of medication to the nebulization catheter 22 at predetermined times for predetermined intervals is also contemplated. In one embodiment, the pump may include a heating mechanism to heat the fluid to a controlled temperature prior to delivery to the fluid line 26 and nebulizing catheter 22.

The gas and fluid lines 18, 26 may be constructed from disposable polyvinyl chloride tubes, although in other embodiments any suitable materials may be used. For example, the tubing may be made of a silicone material that is reusable. The diameters of the tubes may be varied depending on flow rate requirements and any regulations that are in force. Also, the inner diameter of each of the tubes may be different from each other. A filter (not shown) may be located in each of the tubes used for the gas lines 18 to provide a particulate barrier. In one embodiment, the filter may be a glass-fiber hydrophobic filter that provides a particulate barrier of approximately 0.2 microns and operates at a ninety-nine percent rate of efficiency. In other embodiments any number of commonly used filters, with different filtering capabilities, may also be used.

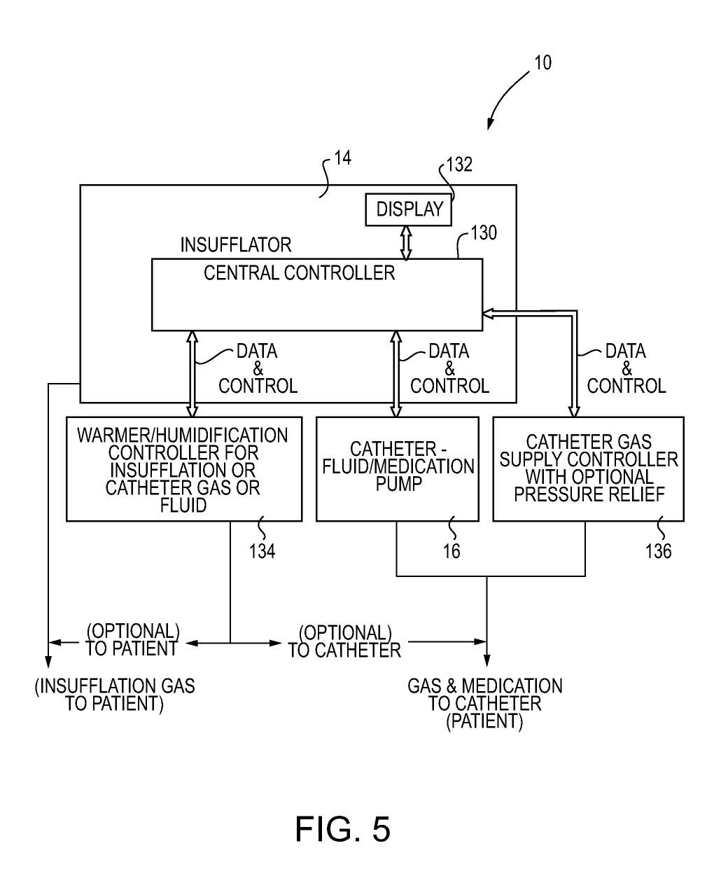

The pressure relief valves (PRV's) 24, 25 associated with the insufflation and aerosolization gas supplies, respectively, may be located within the gas supply lines 18 or the catheters 20, 22. In other embodiments the valves 24, 25 may each be a discrete valve such as commonly available from Pneutronics, a division of Parker Hannifin Corporation of Cleveland, Ohio. Any of a number of types of valves may be used. For example, the valve may be operated electrically, pneumatically, or hydraulically. In other embodiments, the valve may be a mechanical pressure relief valve preset to relieve pressure once a preset maximum has been reached. For example, when the pressure of the insufflation gas reaches a preset pressure, a spring operated valve opens and provides pressure relief. Preferably, the valve is operated by a signal generated by a controller associated with the electronics of the insufflator. An example of such a controller is contained within the control circuitry of the Northgate OMNIFLATOR 6620 Insufflator, and an example of such a valve is a pinch valve. The signal is generated via feedback due to the monitoring of flow restriction or back pressure sensed by a central controller 130 (See FIG. 5). The monitoring of the pressure of the insufflation gas is accomplished via a pressure transducer (not shown) in the controller 130 that monitors the pressure.

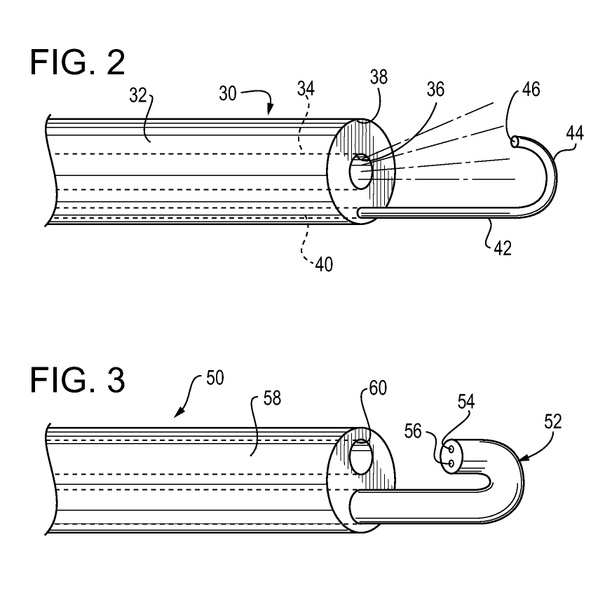

The nebulizing catheter 22 preferably includes a combination of at least one fluid lumen and at least one gas lumen oriented to mix the gas and fluid to generate an aerosol mist inside the peritoneum. Any of a number of nebulizing catheters may be utilized, such as those described in U.S. Pat. No. 5,964,223, issued Oct. 12, 1999 and entitled “Nebulizing Catheter and Methods of Use and Manufacture”, the entirety of which is incorporated by reference herein. Some examples of nebulizing catheters are shown in FIGS. 2-4.

FIG. 2 shows a nebulization catheter 30 with a distal end that can be located inside of a peritoneum via a trocar. The nebulization catheter 30 has a coaxial tubular arrangement with an outer tube 32 surrounding an inner tube 34 so that a fluid delivered from a distal liquid orifice 36 of the inner tube 34 is nebulized by the flow of a pressurized gas delivered in a distal direction from the annular region between the inner and outer tubes at the distal orifice 38 of the outer tube 32. In addition, another lumen 40 extends through the shaft of the nebulization catheter 30. This additional lumen 40 connects to a distal tubular extension 42. The tubular extension 42 extends distally of the distal end of the nebulization catheter 30. A distal end 44 of the distal tubular extension 42 curves back on itself so that a distal orifice 46 of the tubular extension 42 is oriented in a proximal direction back at the orifices 36 and 38 of the inner and outer tubes.

The additional lumen 40 also carries a pressurized gas which is directed in a proximal direction by the orifice 46 against the direction of the aerosol plume generated by the gas and liquid exiting the orifices 36 and 38. The gas from the additional lumen 40 presents a counterflow to the gas from these orifices thereby slowing down the velocity of the particles generated from these orifices. In a preferred embodiment, the distal tubular extension 42 may be formed of a suitable material such as stainless steel needle stock.

FIG. 3 shows another embodiment of a nebulizing catheter 50 that incorporates a counterflow arrangement. Like the embodiment described above, in this embodiment the nebulizing catheter 50 may be positioned in a trocar. The nebulization catheter 50 has a distal section 52 that curves back on itself. The nebulization catheter 50 has distal orifices 54 and 56 that generate a plume of nebulized particles in a reverse, i.e. proximal, direction. Also located in the nebulization catheter 50 is another lumen 58 for carrying a pressurized gas. The additional lumen 58 has a distal orifice 60 oriented in a distal direction. The distal orifice 60 of the additional lumen 58 is aligned with respect to the distal orifices 52 and 54 of the nebulization catheter 50 so that the flow of gas from the additional lumen 58 slows down the velocity of the nebulization plume generated from the nebulization catheter 50. The aerosol plume generated by the nebulization catheter reverses direction and is delivered to the peritoneum carried by the flow of gas from the additional lumen 58.

In another embodiment of a nebulization catheter arrangement, the catheter may include three lumens, two gas and one liquid, where the second of the two gas lumens is utilized to sense pressure and/or provide pressure relief to the body cavity.

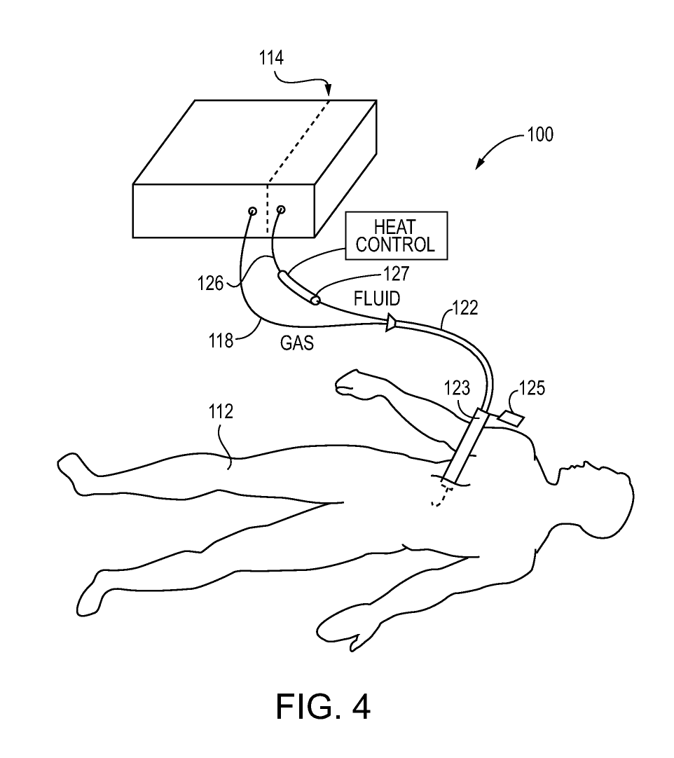

Referring to FIG. 4, an alternative embodiment of the system 100 is shown. In this embodiment, the system 100 provides both the insufflation gas and the aerosolization gas through a single gas line 118 that is routed through the nebulization catheter 122 via the trocar 123 or other needle inserted into the patient 112. A combined insufflator/pump 114 provides both the insufflation gas and the fluid through the nebulizing catheter 122. The fluid is provided along a fluid line 126 that may pass through an optional heating sleeve 127 controlled by a heater controller 128 to warm the fluid to a desired temperature. In an alternative embodiment, the fluid heating mechanism may be integral with the pump or provided by an in-line heater. In another embodiment, where the pump is a syringe pump for controlling fluid discharge from a removable syringe, heat may be supplied to the fluid using syringe heater tape available from Watlow Electric Manufacturing Co. of St. Louis, Mo. The heater may be controlled through a central controller at the combination insufflator/pump 114. The temperature of the fluid is preferably adjusted such that heat loss in the remaining path to the body cavity is accounted for so the fluid is within the desired temperature range as it enters the body cavity. A relief valve mechanism 125 is provided to control the gas pressure so that the gas pressure in the peritoneum or other body cavity is maintained at a desired level. The pressure relief valve 125 may be integrated with the trocar or may be a separate relief valve mechanism positioned along the gas line 118 or in the insufflator. As illustrated in FIG. 4, the system 100 may include combined or separate gas and fluid sources. Additionally, the system may work through a single trocar 123 or through separate trocars as is illustrated in the embodiment of FIG. 1.

As shown in FIG. 5, the system 10 of FIG. 1, is preferably controlled by a central controller 130 which may be integral with, or separate from, the insufflator 14. The insufflator may also include a display 132 for simultaneously or selectively displaying one or more of the parameters managed by the central controller 130. Preferably, the central controller 130 is in communication with each of the components of the system, whether integrated with the insufflator 14 or discrete. Thus, the central controller 130 may monitor and adjust the temperature and humidification control of the insufflation and catheter gas via the gas controller 134, the operation of the pump 16 providing medication to the catheter and the controller 136 connected with the pressure relief valve or valves on the insufflation gas supply and/or the catheter gas supply. One or more of the controllers 130, 134, 136 and the display 132 may be integrally formed with, or independent of, the insufflator 14. The display may be provided with one or more standard interface buttons, or a touch screen capability. Any of a number of communication protocols and formats may be used between the central controller 130 and any of the integrated or discrete controllers.

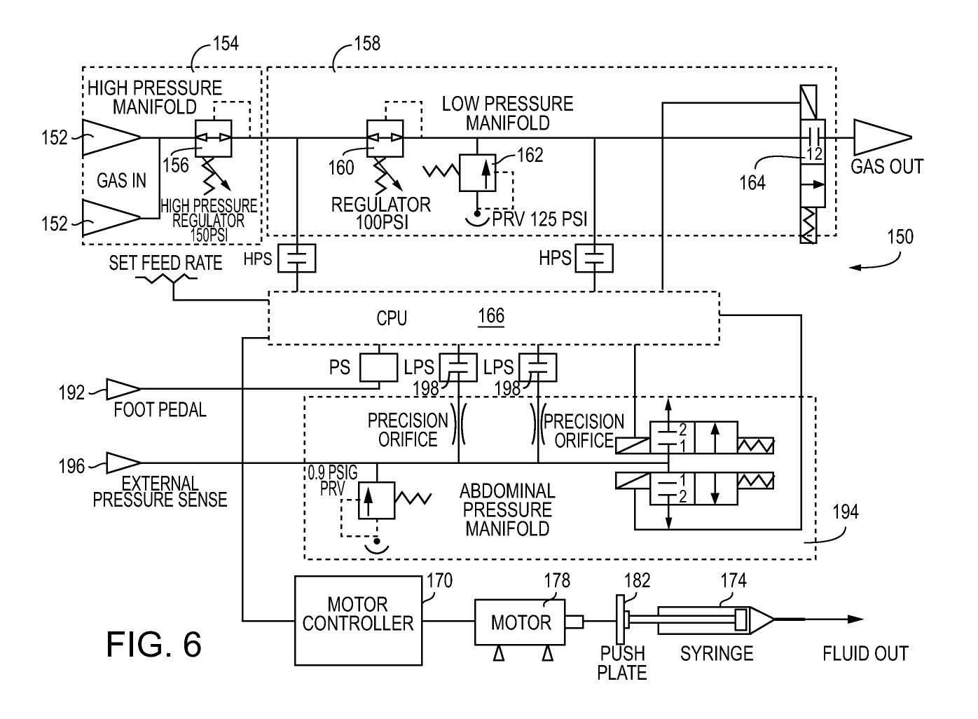

A more detailed diagram of an embodiment of a regulated liquid and gas dispensing controller 150 incorporating a syringe pump, independent CPU and optional active pressure relief mechanism as shown in FIG. 6. The controller 150 combines insufflator and pump controller tasks. In one embodiment, the controller is preferably configured in a high pressure, low flow arrangement that differs from the typical low pressure, high flow arrangement of insufflators generally. Insufflation gas from a high pressure gas source, such as pressurized bottled gas, is connected at the gas inputs 152. A high pressure manifold 154 regulates the pressure from the initial high pressure source, in which gas can be at a pressure in the range of 2000 pounds per square inch (p.s.i.), and reduces the supply pressure through a high pressure regulator 156. In one embodiment, the high pressure regulator 156 reduces the received gas pressure to approximately 150 p.s.i. Any of a number of types of high pressure regulators may be used.

The pressure of insufflation gas supplied to a patient generally needs to be at a lower pressure and so the gas from high pressure manifold at, for example, 150 p.s.i. is then processed through a low pressure manifold 158. The low pressure manifold includes a low pressure regulator 160 configured to further reduce the gas pressures. In this example, the gas pressure is reduced from 150 p.s.i. to 100 p.s.i. This pressure translates to a flow rate of 2-3 liters per minute actually introduced to the body cavity due. The pressures discussed above are merely presented as examples and the various pressure settings in the high and low pressure manifolds may be user adjustable, or may be preset at the manufacturer with no manual settings necessary, at any of a number of pressures. The low pressure manifold also includes a passive pressure relief valve (PRV) 162 set to mechanically release pressure above a predetermined threshold which, in this example, is 0.9 pounds per square inch gauge (p.s.i.g.). An electrically controllable output valve 164 meters the gas output sent on to a catheter. Pressure monitor lines connect a central processor (CPU) 166 to the low pressure manifold via high pressure sensors 168. When used in an insufflator arrangement, at least one of a passive pressure relief valve 24 (See FIG. 1) at the patient may be used to control the pressure introduced to the patient, or the optional active pressure controller 194, described in more detail below, may be utilized. The syringe pump motor controller 170 is also controlled by the CPU to meter the amount of fluid provided to a patient.

An actuator 192 may be connected with the controller 150 to initiate one or more actions by the controller 150. For example, the actuator 192 may send a signal to the CPU 166 that will start or stop the production of insufflation gas, the dispensing of fluids or other activities. In one embodiment, the actuator 192 may be a foot pedal or some other form of actuator that allows a medical practitioner to keep both hands free. Push buttons, levers, touch-screens or any of a number of actuation input means are also contemplated.

An optional portion of the regulated liquid and gas dispensing controller is an active pressure controller 194 that, in addition to the mechanical, passive pressure relief valve 162, can provide a mechanism for limiting pressure supplied to the patient. Although optional, the active pressure controller 194 can provide more precise pressure control by taking a pressure measurement supplied from a sensor via an external pressure sense line 196 at the patient's body and allowing the CPU 166 to actively regulate the pressure. Pressure data may be provided to the CPU 166 by way of low pressure sensors 198. The active pressure controller can reduce the pressure supplied to the patient through one or more active pressure relief valves electrically controllable by the CPU.

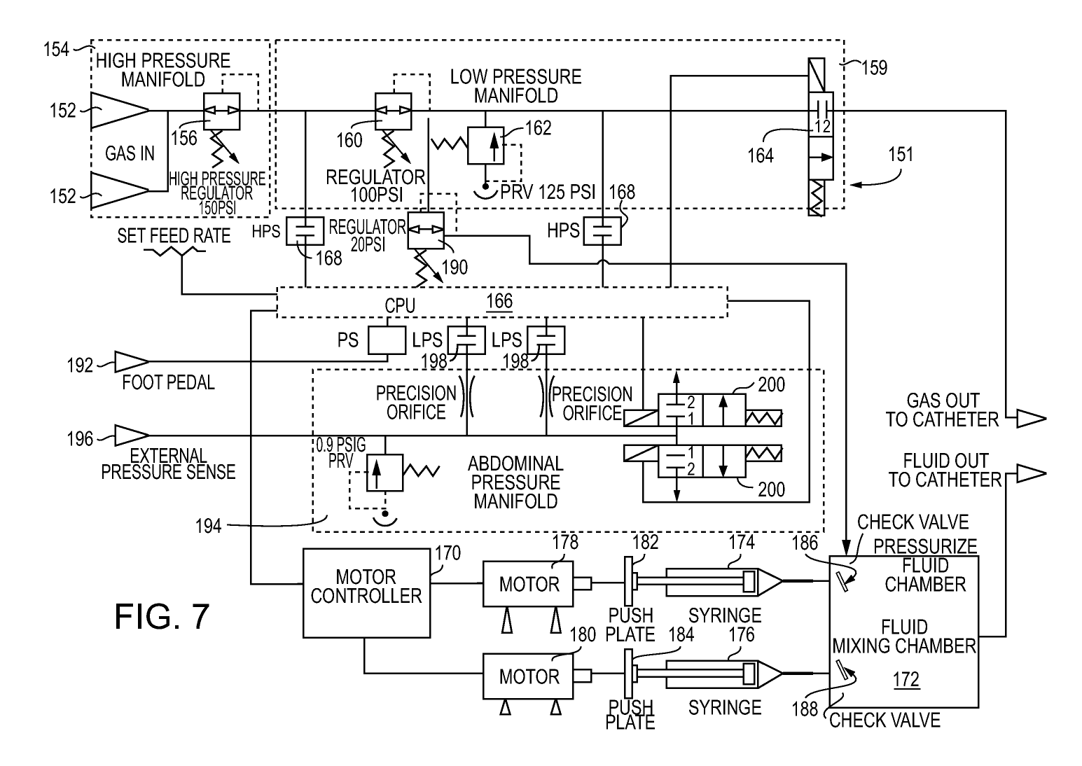

Some operative and post-operative therapies may require a mixture of more than one fluid. The fluid mixture can be achieved through a number of minor modifications. One embodiment of a regulated liquid and gas dispensing controller 150 with multiple fluid sources is illustrated in FIG. 7. In the embodiment of FIG. 7, a mixture of fluids is provided by a configuration of the regulated liquid and gas dispensing controller 151 that utilizes a fluid mixing chamber 172 to mix different fluids provided by separate syringes 174, 176. The motor controller 170 interprets instructions from the CPU 166 to activate the separate motors 178, 180 linked to push plates 182, 184 to engage the respective syringes 174, 176.

Upon a signal from the CPU 166 and motor controller 170, each motor 178, 180 will move its push plate a certain metered distance and cause the syringe to eject a measured amount of fluid into the fluid mixing chamber 172. Each motor 178, 180 may be instructed to move the same or different amount depending on the desired mixture of fluids. Check valves 186, 188 may be included on the input ports of the fluid mixing chamber as added protection against back flow into the same or different syringe. In order to provide sufficient pressure to eject the mixture of fluid from the fluid mixing chamber, such as a 20 p.s.i. or other low pressure regulator, supply of gas from the low pressure manifold 159 is taken after the low pressure regulator 160 and further processed through a mixing chamber pressure regulator 190 down to, in this example, 20 p.s.i. The gas is then transmitted to the fluid mixing chamber to propel the mixed fluid to the catheter for nebulization in a body cavity, for topical application or other application. Using this embodiment, the different fluids can be administered in combination or consecutively, where a single fluid is sent through, and evacuated from, the mixing chamber before the next fluid is dispensed.

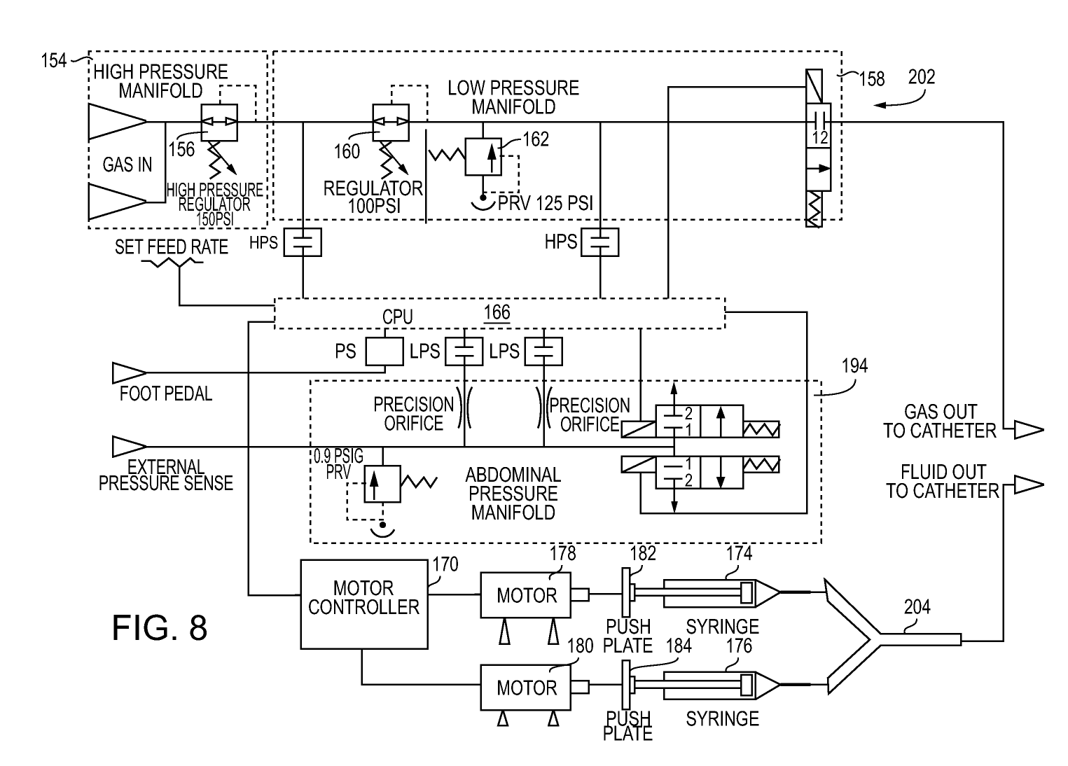

Another embodiment of a controller 202 configured for fluid mixture is shown in FIG. 8. In this embodiment, all the same components as in FIG. 7 are identified with the same reference numbers. The embodiment of FIG. 8 differs from that of FIG. 7 in that a passive y-tube 204 replaces the fluid mixing chamber 172 and fluid mixing chamber regulator 190 of FIG. 7. Thus, the mixing of fluids and delivery of the fluid from the syringes 174, 176 to the catheter takes place using the force of the push plates 178, 180 on the syringes. The different fluids may be combined in the y-tube by simultaneously dispensing the fluids from the syringes. Alternatively, the fluids may be dispensed consecutively or at widely spaced time intervals depending on the application.

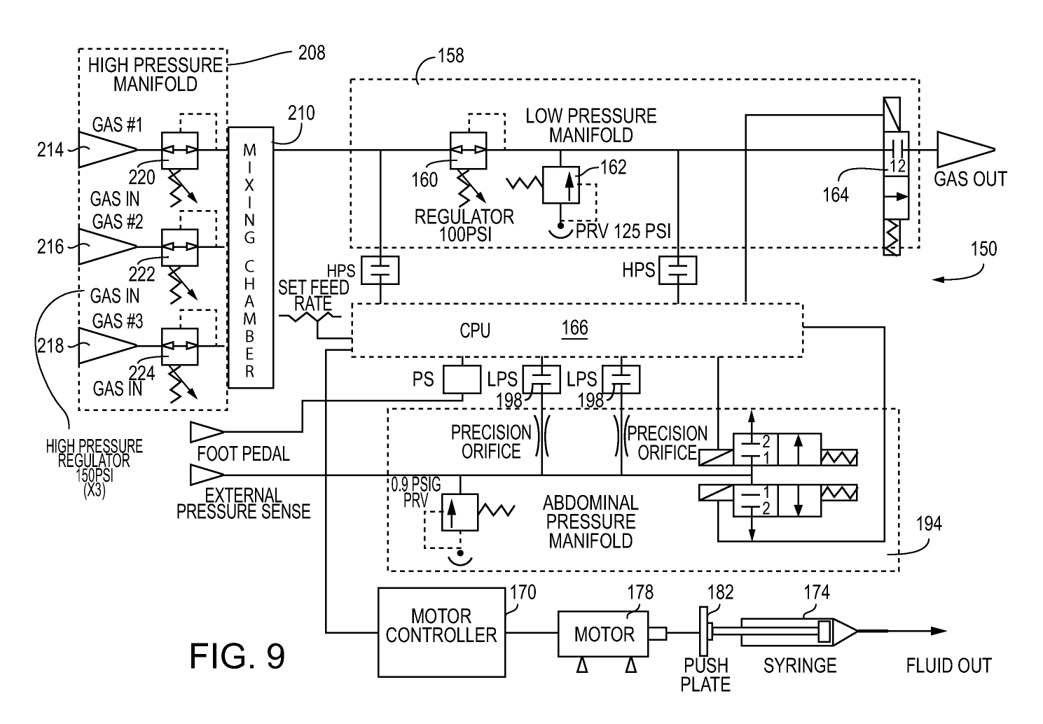

In addition to providing configurations of a controller for providing a single type of fluid, or multiple types of fluids, embodiments of the present invention include configurations and methods for accommodating multiple different gases. In one embodiment, shown in FIG. 9, a modified high pressure manifold 208 and mixing chamber 210 in a controller 212 may be used to replace the high pressure manifold 154 of FIGS. 6 and 7. The remaining components of the controller 212 in FIG. 8 identical to those in FIGS. 6 and 7 retain the same reference numerals for clarity. Using the controller 212 of FIG. 8, a mixture of different insufflation gases 214, 216, 218 are processed in respective high pressure regulators 220, 222, 224 to bring their pressures down to a lower pressure, 100 p.s.i. in this example, more easily managed by the mixing chamber 210. The mixing chamber, an example of which is disclosed in U.S. application Ser. No. 10/829,485 incorporated above, combines substantially even amounts of the gases into a mixture that is then processed through the low pressure manifold 158 as previously described. Examples of applications for mixed gas insufflation include the prevention of acidosis through the addition of oxygen to the insufflation gas, the reduction of post-operative pain through the addition of helium or oxygen, and other such applications.

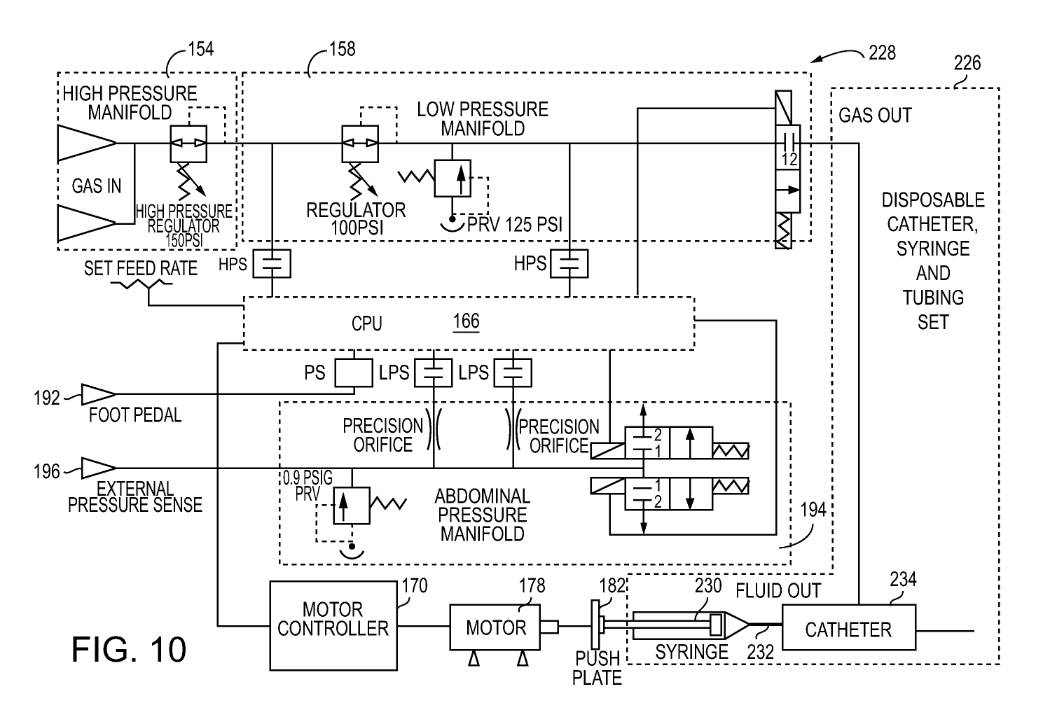

In another embodiment, the fluid pump assembly of the regulated liquid and gas dispensing controller, which includes the motor controller 170, motor 178, and push plate 182, may be adapted to work with a disposable catheter, syringe and tubing set 226. As shown in FIG. 10, the set allows for increased isolation of any fluid from contact with the rest of the controller 228. This is achieved by including a direct syringe 230 to tube 232 to catheter 234 connection rather than a separate, fixed syringe holder that encloses a syringe on the interior of the holder and attaches a tube to the outside of the syringe holder where fluid contacts a conduit built into the holder between the syringe and catheter or tube. To provide further isolation from contamination, the tube 236 or other conduit from the gas outlet of the low pressure manifold to the catheter is also preferably part of the set 226.

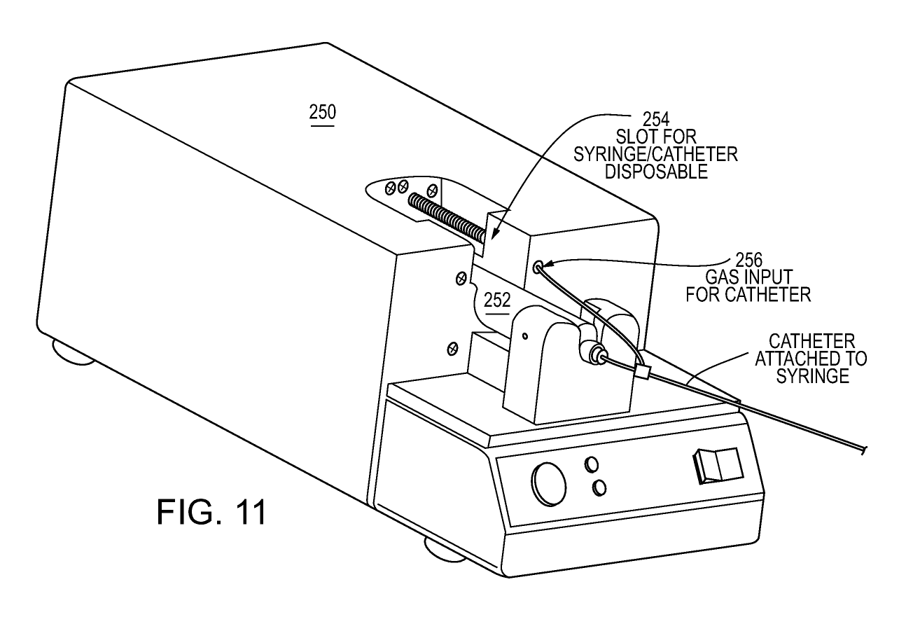

FIG. 11 discloses a perspective view of a syringe pump assembly 250 having a receiving slot 254 for a disposable syringe/catheter/tubeset. The catheter (not shown) may be preassembled as attached to the syringe 252 and replaceably insertable with the syringe as the syringe is place into the receiving slot 254 the syringe pump assembly, or the catheter may be separated from the syringe and still directly attached to the syringe without any intervening, non-disposable lumen. The syringe pump assembly 250 may be in communication with a remote processor or contain its own processor for managing operation of the syringe pump and any gas supply that may also be incorporated. In one embodiment, the housing of the syringe pump assembly may also contain pneumatics for supplying insufflation/catheter gas so that all the elements of the regulated liquid and gas dispensing controller discussed previously are maintained in a single housing. In this embodiment, a catheter gas input port 256 may be integrally formed in the assembly so that a gas may be provided to the catheter attached to a syringe mounted in the assembly. A passive pressure relief valve located at the patient may be used to control insufflation pressure or the assembly 250 may contain an active pressure relief valve. In the embodiment where an active pressure relief valve is incorporated, a pressure sense port for communicating with a pressure sense line from a patient would also be integrated into the housing of the assembly.

Utilizing the integrated system or separate system components described above, a method of providing a substance, such as a nebulized medication, to a body cavity during a minimally invasive procedure is now described. Although a laparoscopic procedure is specifically identified below, the applications of medication using this system can include administration of nebulized substances onto or into specific organs and lumens in the body, as well as topical applications. Additionally, the systems and methods described herein are applicable to minimally invasive procedures generally. In many normal laparoscopic procedures, such as for gall bladders, hernias, bowl resections and etc., a patient is placed in the prone position and sedated. A verres-type needle is placed in the patient to transport gas to the patient and this verres needle is connected to the insufflator to pump up the peritoneum. One suitable verres needle or, more generally, insertion device is disclosed in U.S. application Ser. No. 09/841,125, filed Apr. 24, 2001 and published on Dec. 5, 2002 as Pub. No. US 2002/0183715, the entirety of which is incorporated herein by reference. The verres needle may then be removed and a trocar inserted through the needle hole already made, while maintaining a supply gas in the cavity. Using the opening provided by the trocar, an endoscope is inserted so that a physician may see inside the body. At this point, several other smaller trocars may be inserted into the body for instruments to be used as needed for the particular procedure.

Utilizing the system described above, the insufflation gas is preferably heated and humidified, and an appropriate medicament treatment is applied. For example, to avoid adhesion problems which may often occur in laparoscopic procedures, an aerosol can be provided via the aerosolization catheter to cover the exposed organs and wall of the abdomen. This anti-adhesion treatment may be repeated multiple times during a surgical procedure and be preprogrammed into the central controller 130 of the system. During the procedure, the parameters relating to the delivery of gas and fluid may be displayed and individually controlled. The parameters may include humidity, temperature, pH, volume, rate, pressure, and duration of any of the fluid or gas being injected into the patient. The pH may be adjusted by, for example, the introduction of acid or buffer solutions to the fluid. While any of a number of catheters may be used with the various embodiments of the regulated liquid and gas dispensing controller to apply a medication, or supply both the insufflation gas and a medication, two suitable catheters are disclosed in U.S. Pat. No. 6,379,373, issued Apr. 30, 2002, and 6,165,201, issued Dec. 26, 2000. The disclosure of both of these U.S. patents is incorporated herein by reference.

With the system and method described above, a physician may apply an aerosolized medicament to a distended body cavity that will allow for efficient, safe and effective application of any number of potentially aerosolized liquids which can be used for pain and management (analgesics), infection prevention (prophylactic antibiotics), tissue adhesion (any number of formulations can be used including naturally occurring lubricious medications such as hyaluronic acid, or any number of other medicaments such as heparin, glycerin or glycol medications, or even humidity), and tumor prevention (using targeted or prophylactic chemotherapy drugs or methods) or to control bleeding or blood clotting. Although laparoscopic procedures are specifically discussed above, the systems and methods disclosed herein are contemplated for use in any endoscopic or other minimally invasive procedure.

With reference to targeted or prophylactic chemotherapy, according to another aspect of this invention, the system may be used for general continued, and post-operative applications of a substance by re-instituting an environment in the patient in which subsequent applications of the substance, such as an aerosolized medication, may be administered. This may be accomplished by leaving a port device in the patient after a surgical procedure, or by surgically placing a port in the patient in preparation of a non-surgical treatment regimen. The port may be any device capable of providing a sanitary access point to a body cavity, where the device is a resealable mechanism that attaches to the exterior of the abdomen and the interior wall of the abdomen. One example of a suitable port is an enteral feeding tube port. The port permits the device for applying a substance to the body cavity, in this instance a nebulizing catheter, and the remainder of the system 10 of FIG. 1 to be reconnected to the patient at a later time to apply the substance or other treatment. In one embodiment, the substance may be an analgesic to assist with post-operative pain. In another embodiment, the substance may be an antibiotic for application sometime after surgery to combat infection that may arise.

Alternatively, independently of any post-operative pain or infection, a patient may be provided with such a port for the purpose of allowing an effective chemotherapy treatment. In this situation, a patient would be provided with the port so that the organ or organs affected by a cancer may be directly treated with aerosol treatment customized for that particular patient or tumor. In either situation, post-operative reentry or chemotherapy application, treatment may be accomplished without an endoscope. In some embodiments, an endoscope may be used to allow a medical professional to properly apply the aerosol to the desired region and so that a distal end of a nebulizing catheter may be oriented to provide optimal aerosol placement. During the re-entry into the peritoneum, the pressure relief valve or valves (active or passive) are used to maintain a safe cavity pressure. By maintaining proper pressure within the peritoneum, any additional pressure introduced by the gas used in the aerosolization of the medicine, or pressure from the introduction of fluids or other substances from outside the body cavity may be accounted for.

### Heparin/Hyaluronic Acid

In one embodiment, a method for preventing or minimizing adhesions includes combining a first substance comprising hyaluronic acid (HA) and a second substance comprising heparin to produce a liquid mixture suitable to be injected under pressure using a delivery system such as described above. Although the delivery system may be any of the delivery systems 10, 100 noted above, other delivery systems capable of aerosolizing the liquid mixture for administration in aerosol form in the peritoneal cavity may be used. The delivery of a mixture of the first and second substances is preferred, however the first and second substances may be delivered serially in some instances depending on the surgical procedure and site. The aerosolized mixture is preferably administered upon completion of a surgical procedure, although it can also be applied prior to and/or during a surgical procedure.

The first and second substances are present within the mixture at a ratio of first substance to second substance (HA to heparin) in the range of 1:1 to 1:10 by volume. In other embodiments the ratio may be inverted such that more HA than heparin may be in the mixture by volume. For example, the ratio may be 4:1 HA to heparin. By delivering the mixture in aerosol form, a mixture volume of as little as 15 milliliters (ml) may have the desired anti-adhesion effect. The mixture may comprise concentrations of 1%, 2%, 3% or 4% HA. Additionally, it is contemplated that volumes of no less than 50 units/ml of HA and no more than 500 units/ml of heparin will be used. Preferably, the mixture comprises 2% HA and 100 units/ml of heparin and most preferably 1% HA and 100 units/ml of heparin. 1 unit of heparin is an amount approximately equivalent to 0.02 milligrams (mg) of pure heparin. Unfractionated heparin may be used although low molecular weight heparin is preferred as low molecular weight heparin generally has a half life of 4-5 hours compared to 1-2 hours typical of unfractionated heparin. Heparin derived from tissues of porcine or bovine is preferred although synthetic heparin may also be used instead. In one exemplary embodiment, a pharmaceutical grade of heparin derived from mucosal tissues of animals may be used, such as is available from any of a number of pharmaceutical manufacturers. In one embodiment, the HA used may be NeoVisc® 1% HA available from Stellar Pharmaceuticals Inc.

In one exemplary embodiment, the HA has a molecular weight in the range of 600 kilo Daltons (kDa) to 4,000 kDa, or preferably a range of 1,000 kDa to 2,000 kDa. In the same exemplary embodiment, the heparin may have a molecular weight in the range of 3 kDa up to 30 kDa and most preferably a molecular weight in the range of 5 kDa to 15 kDa. As is known, molecular weights are proportional to viscosities and thus the higher the molecular weight the higher the viscosity for each of these substances. The delivery device lumens and pressure administered may be varied to accommodate the different viscosities. For example, in a nebulizing catheter having 5 gas lumens and a single liquid lumen such as described below, the gas lumens would each have an inner diameter ranging from 0.006+/−0.0005 inches at a proximal end tapering to gas orifices each having a size of 0.003 inches at the distal end of the catheter, and the liquid lumen would have an inner diameter of 0.009+/−0.001 inches at a proximal end tapering to a single liquid orifice having a size of 0.005 inches at the distal end. The pressure applied to the gas lumens may be 60-120 p.s.i. and to the liquid lumen 40-100 p.s.i.

In addition to the two substances of HA and heparin, a third substance such as water, saline, buffer and/or any other fluid may be added to solubilize the first substance and/or second substance, or to reduce the molecular weight of one or both of the first and second substances, if necessary to one or both substances or to the mixture. Other substances which do not chemically interact with HA or heparin may also be added, including certain cancer medications or painkillers such as, without limitation, the pain medication Bupivacaine. Preferably, a volume of 10 ml to 20 ml of Bupivacaine would be added to the mixture or most preferably 15 ml. The administration of Bupivacaine (such as Marcain®) in liquid form has been shown in some studies as reducing post-operative shoulder pain.

The aerosolized liquid mixture preferably comprises particles of an aerodynamic size ranging from 10 microns up to 25 microns, and preferably from 13 microns to 25 microns, and most preferably 15 microns to 20 microns. At these ranges, the aerosolized liquid mixture can expand within the peritoneal cavity in an aerosol cloud formation and remain in suspension for up to 3-5 minutes. This ensures that the peritoneal cavity is properly coated, including the underside of the abdominal wall and organs which during the 3-5 minute period may change position and orientation in the body cavity.

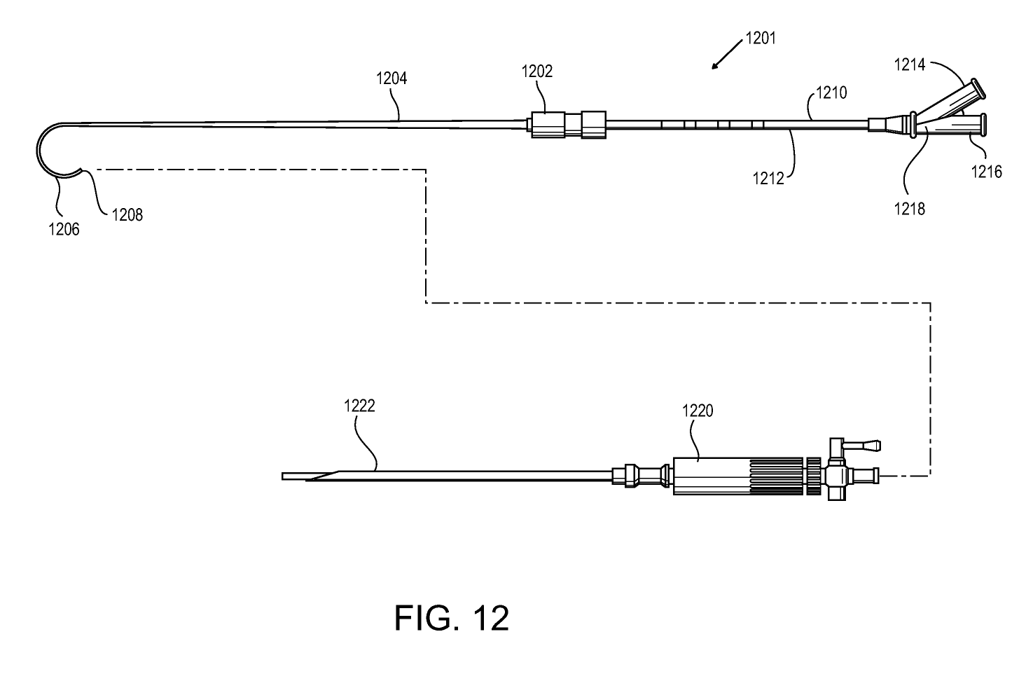

Referring to FIG. 12, an embodiment of one suitable nebulizing catheter 1201 for use in a delivery system that can generate the aerosol size for the HA/heparin mixture discussed above, and can direct the aerosol to desired parts of a peritoneum, is shown. The catheter 1201 consists of a tapered section 1204 toward the distal end and a proximal portion 1210 accessible for axial adjustment of the catheter. The proximal portion 1210 connects with a catheter hub 1218 connected with a catheter gas port 1214 and a catheter liquid port 1216. In one embodiment, the catheter 1201 includes a flexible, pre-shaped tip 1206 that is curved or angled such that retraction of the catheter 1201 through a needle, such as the needle 1222 of the introducer apparatus 1220, will cause the pre-shaped tip 1206 to change its angle orientation.

The catheter may be formed of any of a number of flexible materials, such as nylon. A shaped spring wire or other type of resilient reinforcing shaft may be inserted or embedded in the full catheter shaft and/or tip area to create the intended shape. Alternatively, the polymer or catheter material may be selected and manufactured to impart a resilient shape or curve without the need for a reinforcing member. One suitable type of catheter adaptable for use in the system 10 is described in U.S. Pat. No. 5,964,223, the entirety of which is incorporated herein by reference.

As shown in the exploded view of FIG. 12, the catheter 1221 may be positioned in an introducer needle that can be used to pierce an abdominal wall of the patient. A suitable introducer and catheter is further described in U.S. Pat. No. 7,914,517, the entirety of which is hereby incorporated herein by reference. The introducer apparatus 1220 releasably connects with the introducer needle 1222 on one end and may include a joint or other mechanism for permitting rotation so that the pre-shaped tip 1206 can be oriented to point in different directions in a body cavity. Alternatively, the catheter 1221 may simply be introduced into the patient via a standard needle such as a Verres needle. When retracted or inserted in either the introducer apparatus or just a basic Verres needle, the flexible tip bends against the distal end of the needle and alters its orientation when a physician pulls out or inserts the catheter 1221 such that the tip 1206 is straightened or allowed to return to its predetermined shape as different portions of the tip contact the distal end of the needle.

The proximal portion 1210 of the catheter 1201 may include one or more catheter markings 1212 to assist a physician in gauging how deeply the catheter tip 1206 is inserted, and therefore what angle the tip 1206 may be at, in the body cavity. The catheter markings are spaced apart at even intervals in one embodiment. Catheter markings 1212 may be spaced apart at uneven intervals in other embodiments. The catheter markings may consist of bands of the same or different colors, may include indicia indicative of an insertion depth or orientation of the distal end of the catheter, or may consist of one or more different texture regions. The texture regions may be uniform or may consist of differing shapes or configurations (e.g. raised or recessed regions). Any of a number of other forms of indicia is also contemplated.

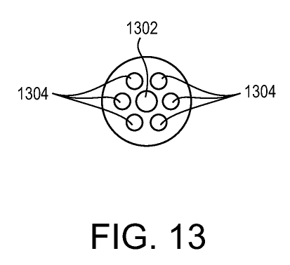

The catheter 1201 may be constructed with a single lumen or multiple lumens. In one embodiment, a multiple lumen arrangement may be used, where more than one of the lumens may be dedicated to a nebulizing gas and one of the lumens is dedicated to carrying a mixture of HA and heparin. FIG. 13 illustrates one of a number of liquid and gas orifice configurations at the nozzle 1208 of the pre-shaped tip 1206 on the catheter 1221. In this embodiment, a central liquid orifice 1302 is encircled by a plurality of gas orifices 1304. The gas and liquid orifices may be aligned such that the gas and liquid introduced into the catheter at the gas port and liquid port interact upon exiting through the gas and liquid orifices to form an aerosol. In other embodiments, different numbers and arrangements of orifices may be used. For example, in one preferred embodiment, an arrangement of five gas lumens and five corresponding gas orifices 1304 may surround a central liquid orifice 1302. Other numbers of gas orifices, including greater than or less than five orifices in other implementations. Additionally, a single gas lumen with a single gas orifice may be used. In each of the alternative arrangements, the differing number of lumens or orifices are suitable if the lumen and or orifice sizes are configured to generate the desired nebulized mixture of HA and heparin concentrations discussed above with particles having an aerodynamic size ranging from 10 microns up to 25 microns, and preferably from 13 microns to 25 microns, and most preferably 15 microns to 20 microns.

In one embodiment, a multiple lumen nebulizing catheter such as catheter 1221 may be used with the mixture of HA and heparin as discussed above to achieve a particle size in the range of 10 to 25 microns using multiple gas lumens. For example, an embodiment of a catheter may include 5 gas lumens, each with an inner diameter of 0.006 inches +/−0.0005 inches at the proximal portion 1210 of the catheter 1201, and a liquid lumen having an inner diameter of 0.009 inches +/−0.001 inches at the proximal portion 1210. These lumens may be tapered down in the tapered section 1204 to inner diameters of 0.003 inches for the gas orifices 1304 at the nozzle 1208 at the end of the pre-shaped tip 1206 and to an inner diameter of 0.005 inches at the liquid orifice at the end of the liquid lumen. As noted above, the liquid lumen diameter, and orifice diameter, may need to be changed to accommodate different viscosity (e.g. different molecular weight) mixtures of HA and heparin to achieve the flow rate and particle size of 10-25 microns (or the other noted ranges) that are desired to generate an aerosol mist that can stay suspended for the desired 3-5 minute period of time. Also, in other embodiments, a nebulizing catheter may be used with lumens that are not tapered but that are sized to generate the desired particle size (e.g. particles of the HA and heparin mixture noted above having an aerodynamic size ranging from 10 microns up to 25 microns, and preferably from 13 microns to 25 microns, and most preferably 15 microns to 20 microns).

As discussed above, a method and apparatus for creating a medicated atmosphere of a mixture of hyaluronic acid and heparin in an organ or body cavity has been disclosed. The method permits a creation of an aerosol cloud allowing for the deposition of a substance comprising a medicament on all or a selected number of interior surfaces. Preferably, the cloud is of particle sizes in the range of 10-25 microns, more preferably in the range of 13-25 microns, and most preferably in the range of 15-20 microns, that can stay suspended for a period of time, such as 3-5 minutes, to allow the aerosolized mixture to deposit over that time on a dynamically changing topology of exposed regions inside a body cavity. The apparatus for depositing the desired mixture may include an aerosolization catheter that can be manipulated during use, a device for the introduction of the aerosolization catheter, a medication delivery system linked to a control means for the control of rate, amount, and time of delivery of the medication, a system for providing pressure control of a gas to the catheter which is also controlled as to pressure, timing and rate of gas flow, a means for monitoring and relieving the pressure of the gas, alone or in conjunction with an insufflator, and a means of integrating the control of the various gas and fluid supplies for complete system control. Additionally, means for reentering the peritoneum or organ post-operatively to recreate a medicated environment for a post-operative treatment is disclosed. In different embodiments, insufflation and nebulization may both be performed through a single gas lumen in a catheter, or multiple gas lumens, using the same regulated liquid and gas dispensing controller.

It is intended that the foregoing detailed description be regarded as illustrative rather than limiting, and that it be understood that the following claims, including all equivalents, are intended to define the scope of this invention.

We claim:

1. A system for providing a controlled environment in a body cavity of a patient, the system comprising:
a source of pressurized gas;
a source of liquid;
a nebulizing catheter comprising:
at least one liquid lumen and at least one gas lumen;
the at least one liquid lumen in communication with the source of liquid and the at least one gas lumen in communication with the source of pressurized gas;
the nebulizing catheter having a distal end positionable in a body cavity;
the at least one liquid lumen and the at least one gas lumen oriented at the distal end to mix the gas and liquid to generate an aerosol inside the body cavity to cover exposed organs and a wall of an abdomen; and
wherein the liquid comprises a mixture of hyaluronic acid and heparin in a ratio by volume in the range of 1:1 to 1:10 of hyaluronic acid to heparin.

2. The system of claim 1, wherein the hyaluronic acid in the mixture comprises no less than 50 units per milliliter.

3. The system of claim 1, wherein the heparin in the mixture comprises no more than 500 units per milliliter.

4. The system of claim 3, wherein one unit of heparin comprises approximately 0.02 milligrams of pure heparin

5. The system of claim 4, wherein mixture comprises 1% hyaluronic acid and 100 units per milliliter of heparin.

6. The system of claim 4, wherein mixture comprises 2% hyaluronic acid and 100 units per milliliter of heparin.

7. A system for providing a controlled environment in a body cavity of a patient, the system comprising:
a source of pressurized gas;
a source of liquid;
a nebulizing catheter comprising:
a liquid lumen and a plurality of gas lumens;
the liquid lumen in communication with the source of liquid and the plurality of gas lumens in communication with the source of pressurized gas;
the nebulizing catheter having a distal end positionable in the body cavity;
the liquid lumen and the plurality of gas lumens sized and oriented at the distal end to mix the gas and liquid to generate an aerosol having a particle size in a range of 10 microns to 25 microns inside the body cavity to cover exposed organs and a wall of an abdomen;
a liquid and gas dispensing controller configured to manage delivery to the nebulizing catheter of gas from the source of pressurized gas and liquid from the source of liquid; and
wherein the liquid comprises a mixture of hyaluronic acid and heparin.

8. The system of claim 7, wherein the hyaluronic acid has a molecular weight in a range of 600 kilo Daltons (kDa) to 4,000 kDa.

9. The system of claim 8, wherein, and the heparin has a molecular weight in a range of 3 kDa up to 30 kDa.

10. The system of claim 9, wherein the gas lumens each have an inner diameter of 0.006 inches +/−0.0005 inches in a proximal portion of the catheter, and the liquid lumen has an inner diameter of 0.009 inches +/−0.001 inches at the proximal portion of the catheter.

11. The system of claim 10, wherein the gas lumens each taper to an inner diameter of 0.003 inches at the distal end and the liquid lumen tapers to an inner diameter of 0.005 inches at the distal end.

12. The system of claim 7 wherein the liquid comprises a mixture of hyaluronic acid and heparin in a ratio by volume in the range of 1:1 to 1:10 of hyaluronic acid to heparin.

13. The system of claim 12, wherein the hyaluronic acid in the mixture comprises no less than 50 units per milliliter.

14. The system of claim 12, wherein the heparin in the mixture comprises no more than 500 units per milliliter.

15. The system of claim 14, wherein one unit of heparin comprises approximately 0.02 milligrams of pure heparin.

16. The system of claim 15, wherein mixture comprises 1% hyaluronic acid and 100 units per milliliter of heparin.

17. The system of claim 15, wherein mixture comprises 2% hyaluronic acid and 100 units per milliliter of heparin.

18. The system of claim 9, wherein the liquid lumen and the plurality of gas lumens are sized and oriented at the distal end to mix the gas and liquid to generate an aerosol having a particle size in a range of 13 microns to 25 microns.

19. The system of claim 9, wherein the liquid lumen and the plurality of gas lumens are sized and oriented at the distal end to mix the gas and liquid to generate an aerosol having a particle size in a range of 15 microns to 20 microns.
